(19) United States
(12) Patent Application Publication
(10) Pub. No.: US 2025/0177707 A1
(43) Pub. Date: Jun. 5, 2025

(54) ASSEMBLIES AND ASSOCIATED METHODS FOR RE-CIRCULATION OF BODY FLUIDS INCLUDING THERAPEUTIC FILTRATION OR CONDITIONING

(71) Applicant: BRAINSPACE, INC., Camden, DE (US)

(72) Inventors: Caitlin D.C. MORSE, Woodinville, WA (US); Stephen A. MORSE, Woodinville, WA (US)

(21) Appl. No.: 18/841,949

(22) PCT Filed: Mar. 2, 2023
(86) PCT No.: PCT/US2023/063596

§ 371 Date: Aug. 27, 2024

Related U.S. Application Data
(60) Provisional application No. 63/315,910, filed on Mar. 2, 2022.
(60) Provisional application No. 63/315,930, filed on Mar. 2, 2022.

(51) Int. Cl. A61M 27/00; A61M 1/00; A61M 39/02

(52) U.S. Cl. CPC A61M 27/006; A61M 1/70; A61M 39/0247; A61M 2027/004; A61M 2039/025; A61M 2039/0282; A61M 2202/0464; A61M 2205/3303; A61M 2205/3334; A61M 2205/3606; A61M 2205/50; A61M 2205/7545; A61M 2230/50

(57) ABSTRACT

Body fluid management systems and associated methods for withdrawal and subsequent re-infusion of body fluids, such as cerebrospinal fluid, can be used for therapeutic and/or diagnostic purposes.

### BACKGROUND

### Technical Field

The disclosure relates generally to body fluid management systems and associated methods.

### Description of the Related Art

Extracorporeal therapy in the current art includes the withdrawal and reinfusion of blood. This is done therapeutically at the bedside, such as in hemodialysis and peritoneal dialysis renal replacement therapies. Blood is also withdrawn, processed with an apheresis technique to remove a target element using lab equipment, and then reintroduced. A third case of extracorporeal therapy is that of ECMO, extracorporeal membrane oxygenation. With this approach, blood externally bypasses the heart and lungs, is pumped through a device that removes carbon dioxide and adds oxygen and then returns the blood to the body. However, bodily volumes of other bodily fluids, such as cerebrospinal fluid (CSF), are insufficient for the approaches described in the current art.

A variety of medical conditions involving trauma or diseases of the central nervous system affecting the brain, spinal cord, and adjacent tissues are very difficult to diagnose or treat with existing methods and equipment. For diagnosis, existing lab-based testing requires the removal of CSF samples for in-vitro evaluation. This is limited by the CSF volume reduction that can be tolerated by a patient in a single sitting. Given this, the existing art fails to deliver a timely solution, such as in the case of Leptomeningeal cancer, where low concentration of analytes or biomarkers are present in the CSF. Similarly, there are many rare brain diseases for which existing testing is not cost-effective because of the low prevalence in the population.

When it comes to treatment, the existing art relies on intravenous delivery and offers few options for delivery of therapeutic agents that do not cross the blood-brain barrier. Attempts have recently been made to deliver such therapeutic agents directly into the CSF, thereby bypassing the blood-brain barrier, but the limitations of existing therapeutic and body fluid management systems make this process cumbersome, minimally effective, or dangerous. There are a variety of reasons for this.

The flow mechanisms, flow patterns, and flow pathways from creation, to circulation, and then reabsorption of CSF are poorly understood. It is unclear from the medical literature where in the patient anatomy therapeutic agents should be delivered to maximize benefit for treatment of a particular tumor type or location. The Kelli-Monroe doctrine is a widely accepted explanation of the pressure within the skull, but it falls short in the modern era of fully explaining the range of patients found in the ICU. It was only within the last decade that the glymphatic system was identified.

Introduction of a therapeutic agent also increases the total volume of fluid within the skull, which typically increases intracranial pressure (ICP) in patients with disrupted cerebral autoregulation if total fluid volume and/or pressure are not carefully managed via external drainage. The existing art does not include provision for management of pressure during exogenous fluid introduction. Existing systems for external drainage of CSF do not support simultaneous monitoring, drainage, and infusion, and are notoriously difficult to manage. They are known to be the source of staffing challenges and medical errors in clinical practice leading to patient morbidity and mortality.

### BRIEF SUMMARY

Disclosed are body fluid management systems and associated methods that include the withdrawal of a body fluid and subsequent re-infusion of the same body fluid after conditioning, such as thermal conditioning, filtration, introduction or removal of chemical or biological agents, or other therapeutic process(es). Disclosed body fluid management systems may additionally include in-line measurement and/or detection of analytes, contaminants, and/or analysis of physical characteristics of the fluid for diagnosis or detection of disease, infection, or toxicity.

A body fluid management system configured for recirculation and drainage of a body fluid may be summarized as comprising a patient interface assembly including a first fluid line, a second fluid line, a body fluid collection reservoir, and at least one wearable sensor subassembly, wherein said first fluid line is configured for connection to a first implanted catheter at a first patient access location and said second fluid line is configured for connection to a second implanted catheter at a second patient access location; and a control system assembly including at least one fluid detection subassembly, at least one logic device, and a plurality of flow control mechanisms; wherein said logic device implements an algorithm to control operation of said plurality of flow control mechanisms based on inputs from at least one of said fluid detection subassembly and said wearable sensor subassembly; wherein said plurality of flow control mechanisms are configured to variably withdraw said body fluid from said first patient access location, re-infuse said body fluid at said second patient access location, and drain said body fluid into said body fluid collection reservoir.

Said patient interface assembly may include a third fluid line configured at one end for connection to an exogenous fluid source containing an exogenous fluid. Said patient interface assembly may include a mixing chamber configured for mixing said exogenous fluid and said body fluid prior to re-infusion at said second patient access location. Said wearable sensor subassembly may comprise at least one pressure sensor in direct fluid communication with said body fluid and said control system assembly may be configured for monitoring changes in a pressure of said body fluid. Said wearable sensor subassembly may comprise at least one analyte sensor in direct fluid communication with the body fluid and said control system assembly may be configured for monitoring changes in a concentration of a target analyte in said body fluid. Said wearable sensor subassembly may comprise at least one physiological sensor and said control system assembly may be configured for monitoring changes in a physiological parameter. A method of withdrawal of said body fluid may be gravity-based drainage. Said body fluid may be cerebrospinal fluid.

Said first and second patient access locations may be proximate to a single anatomical marker, and said at least one wearable sensor subassembly may be configured for attaching to the patient proximate to said single anatomical marker. Said anatomical marker may be an external auditory meatus. Said first patient access location may be proximate to a first anatomical marker and said second patient access location may be proximate to a second anatomical marker. Said at least one wearable sensor subassembly may comprise a first wearable sensor subassembly and a second wearable sensor subassembly, said first wearable sensor subassembly may be configured for attaching to the patient proximate to said first anatomical marker and said second wearable sensor subassembly may be configured for attaching to the patient proximate to said second anatomical marker. Said first anatomical marker may be an external auditory meatus and said second anatomical marker may be a lumbar region of the spine.

A body fluid management system configured for conditioning and recirculation of a body fluid may be summarized as comprising: a patient interface assembly including a first fluid line, a second fluid line, at least one wearable sensor subassembly, and at least one body fluid conditioning assembly, wherein said first fluid line is configured for connection to a first implanted catheter at a first patient access location and said second fluid line is configured for connection to a second implanted catheter at a second patient access location; and a control system assembly including at least one fluid detection subassembly, at least one logic device, and a plurality of flow control mechanisms; wherein said logic device implements an algorithm to control operation of said plurality of flow control mechanisms based on inputs from at least one of said fluid detection subassembly and said wearable sensor subassembly; wherein said plurality of flow control mechanisms are configured to variably withdraw said body fluid from said first patient access location, re-infuse said body fluid at said second patient access location, and drain said body fluid into said body fluid collection reservoir.

Said body fluid conditioning assembly may be a thermal conditioning assembly and said thermal conditioning assembly may be configured to adjust a temperature of said body fluid in said second fluid line upstream of said second patient access location. Said thermal conditioning assembly may be configured to increase the temperature of said body fluid. Said thermal conditioning assembly may be configured to decrease the temperature of said body fluid. Said patient interface assembly may include at least one temperature sensor configured to measure the temperature of said body fluid, and said control system assembly may be configured to control operation of said thermal conditioning assembly based on inputs from said temperature sensor. Said control system assembly may be configured to receive body temperature input and adjust an output of said thermal conditioning assembly based on said input. Said body fluid conditioning assembly may be a filtration assembly configured for capturing one or more contaminants from said body fluid. Said body fluid may be cerebrospinal fluid.

A body fluid management system configured for recirculation and drainage of a body fluid may be summarized as comprising: a patient interface assembly including a bi-directional body fluid line, a body fluid collection reservoir, and at least one wearable sensor subassembly, wherein said fluid line is configured for connection to an implanted catheter at a patient access location; and a control system assembly including at least one fluid detection subassembly, at least one logic device, and a plurality of flow control mechanisms; wherein said logic device implements an algorithm to control operation of said plurality of flow control mechanisms based on inputs from at least one of said fluid detection subassembly and said wearable sensor subassembly; wherein said plurality of flow control mechanisms are configured to variably withdraw said body fluid from said patient access location, temporarily store said body fluid, re-infuse said body fluid at said patient access location, and drain said body fluid into said body fluid collection reservoir.

### BRIEF DESCRIPTION OF THE SEVERAL VIEWS OF THE DRAWINGS

FIG. 1 illustrates a body fluid drainage and recirculation management system.

FIG. 2 illustrates a cartridge subassembly of a patient interface assembly.

FIG. 3 illustrates a top view of a wearable sensor subassembly of a patient interface assembly of a body fluid drainage and recirculation management system.

FIG. 4A illustrates a first schematic of a wearable sensor subassembly of a patient interface assembly of a body fluid drainage and recirculation management system.

FIG. 4B illustrates a second schematic of a wearable sensor subassembly of a patient interface assembly of a body fluid drainage and recirculation management system.

FIG. 5A illustrates a fluid detection subassembly and drip chamber with attached filter.

FIG. 5B illustrates a fluid detection subassembly with drip chamber with removeable or replaceable filter.

FIG. 5C illustrates a cartridge subassembly with removeable or replaceable filter.

FIG. 6 illustrates a simplified operational diagram of a drainage control algorithm.

FIG. 7 illustrates a simplified operational diagram of a drainage and recirculation control algorithm.

FIG. 8 illustrates a simplified operational diagram of another drainage and recirculation control algorithm.

FIG. 9A illustrates a first view of a thermal conditioning assembly of a body fluid drainage and recirculation management system.

FIG. 9B illustrates a second view of a thermal conditioning assembly of a body fluid drainage and recirculation management system.

FIG. 10A illustrates a thermal transfer subassembly of a thermal conditioning assembly.

FIG. 10B illustrates a thermal transfer subassembly of a thermal conditioning assembly.

FIG. 11 illustrates a body fluid drainage and recirculation management system with thermal conditioning.

FIG. 12A illustrates a top cutaway view of a wearable sensor subassembly of a patient interface assembly of a body fluid drainage and recirculation management system.

FIG. 12B illustrates a side cutaway view of another wearable sensor subassembly of a patient interface assembly of a body fluid drainage and recirculation management system.

FIG. 13A illustrates a fluid line of a body fluid drainage and recirculation management system.

FIG. 13B illustrates another fluid line of a body fluid drainage and recirculation management system.

FIG. 13C illustrates another fluid line of a body fluid drainage and recirculation management system.

### DETAILED DESCRIPTION

The current disclosure fills unmet needs in the art by describing systems and associated methods that facilitate diagnosis or delivery of therapeutic agents into a living being, and simultaneously monitor relevant physiological parameters (physical movement, fluid pressure and/or flow volume, etc.) and/or manage certain parameters (ICP, flowrate, etc.) within a clinically safe range, maintain and/or drive natural flow patterns of fluid(s) within the body, and/or deliver therapeutic processes (hyper-/hypo-thermic conditioning, filtration, etc.) in an automated or semi-automated fashion. The current disclosure provides these benefits in a configuration that requires minimal clinical expertise to operate, driving broader clinical adoption and ensuring that patients are treated safely, consistently, and with maximum effectiveness. No equipment currently exists that can safely maintain and/or drive the movement of fluid through these pathways. For example, it may be clinically advantageous in certain cases to withdraw CSF from one location (e.g., the spine), alter the withdrawn CSF in some way (e.g., heat or cool the CSF, inject a chemotherapy drug, filter out free-floating cancer cells or blood components, adjust the PH, etc.), then re-infuse the CSF into a different location (e.g., the ventricles).

There is an urgent need for improved therapeutic equipment and methods for earlier stratification of disease and safe and effective delivery of chemotherapy and other therapeutic agents and processes directly into/from the CSF while managing patient ICP within a safe range. Additionally, there is a need for equipment that is able to support clinical research to advance the understanding of medicine with regard to the absorption of exogenous and various body fluids (such as CSF, interstitial fluid, and the like). Finally, there is a need for bedside diagnostic equipment that requires fewer overall tests and a lower volume of fluid to be permanently removed to facilitate an expedited diagnosis. The current art could be significantly improved upon with the introduction of a more effective and efficient CSF screening method to reduce overall time to diagnosis.

Disclosed are body fluid management systems and associated methods that withdraw and subsequently re-infuse one or more body fluid(s) for diagnostic or therapeutic purposes, while providing synchronous tracking and/or control of various physiological parameters. Such systems may additionally advance the scientific understanding of natural flow mechanisms, flow patterns, and flow pathways of various body fluids by providing more detailed tracking and management of physiological parameters than is possible with the current state of the art.

Body fluids in this context may include CSF, blood, interstitial fluid, lymph, and the like.

Physiological parameters in this context may include patient position, orientation, movement, or gait, fluid pressure (such as ICP), fluid temperature, opacity, particulate count, flowrate or drainage volume; and any other related parameters useful in the therapeutic treatment of various medical conditions including brain tumors and metastases, leptomeningeal cancers, Parkinson's Disease, Alzheimer's disease and related dementias, and the like.

Given the physics of pressure created by liquid in a closed system, patient position is a key variable that the current disclosure illuminates. The pressure in the lateral ventricle when a patient is lying down is not the same as when a patient is sitting up. A patient lying flat with typical anatomy has approximately consistent pressure throughout the central nervous system, from the lumbar region of the spine to the skull. When that same patient is standing upright, the pressure in the lumbar spine region is drastically higher, caused by the fluid column of the spine. The pressure in the lateral ventricle can be slightly lower than when lying flat depending on head position. While many studies will lay a patient flat (parallel to the floor) for a particular calculation such as opening lumbar pressure or cerebral perfusion pressure, this position is often temporary. The current disclosure allows for the characterization of position and movement relative to drainage while the recirculation features enable a variety of clinical evaluations to be conducted with the same patient population. The current disclosure also supports evaluation of chronic patients during activities of daily living relevant to the behavior and settings on implanted shunts.

The disclosed body fluid management systems may comprise one or more therapeutic assemblies for achieving the desired therapeutic purpose(s).

The disclosed body fluid management systems may comprise one or more diagnostic assemblies for detection and/or measurement of certain analytes, contaminants, or toxins in the target body fluid, such as the presence and/or concentration of bacteria, viruses, proteins, chemicals including exogenous compounds, elements, hormones, or other biomarkers.

Therapeutic or diagnostic assemblies and associated processes may include thermal conditioning for hypothermic or hyperthermic therapy, detection, capture, and/or removal of deleterious compounds or foreign contaminants such as bacteria (or bacterial biproducts), viruses, cells (such as cancerous cells), proteins (such as tau proteins or proteins associated with the presence of cancerous cells or congenital disease), amyloid-beta peptides, prions, chemical compounds or molecules, chemical elements (such as heavy metals), hormones, biomarkers, glucose, lactate, pyruvate, gamma-aminobutyric acid (GABA), glutamate, neurotransmitters, other body fluids (such as blood contaminating CSF), exogenous compounds (such as pharmaceuticals); introduction of therapeutic agents such as chemotherapy drugs, saline, non-metabolized sugar solution, etc., and the like.

In certain embodiments, the disclosed system may comprise a patient interface assembly and a control system assembly. An embodiment of the disclosed system comprises a disposable patient interface assembly and a durable control system assembly. In other embodiments, the disclosed system may be fully durable or fully disposable.

In certain embodiments, the control system assembly may comprise one or more pump mechanisms for positive displacement of one or more fluids, of which one or more may be body fluid(s) and/or exogenous fluid(s).

In certain embodiments, a patient interface assembly may comprise fluid lines for patient connection at two or more access locations for withdrawal of one or more body fluid(s) from one or more location(s) and re-infusion of body fluid(s) and/or exogenous fluid(s) at one or more location(s).

In certain embodiments, the disclosed system may be configured to recirculate one or more body fluid(s) back into the body. In certain embodiments, the disclosed system may be configured to infuse one or more exogenous fluid(s) into the body. In certain embodiments, the disclosed system may be configured to mix one or more withdrawn body fluid(s) with one or more exogenous fluid(s) or compound(s) and infuse the mixture into the body.

In certain embodiments, the disclosed system may be configured to transfer CSF from one anatomical location to another for diagnostic purposes, such as for the detection of CSF leaks. CSF leaks are a condition where fluid is escaping through a tear or hole in the dura. While imaging is currently used to diagnose CSF leaks, many suspected idiopathic leaks are hard to identify or locate. Therefore, the current disclosure offers an alternative method of detection by intentionally increasing or decreasing CSF pressure and/or volume at various locations along the cerebrospinal column, especially on either side of a suspected region, and recording changes in physical parameters (e.g., pressure over time, pressure response to positional changes, etc.) or patient symptoms (e.g., headache, balance, gait, etc.).

In certain embodiments, the disclosed system may be configured to remove CSF from an anatomical location, store it temporarily extracorporeally, and then return it to the same anatomical location for diagnostic benefit of the fluid reduction itself or as a means to analyze extracorporeally more fluid than would otherwise be possible to remove for analysis.

In certain embodiments, the disclosed system may be configured to provide therapeutic drainage of one or more body fluid(s).

In certain embodiments, the disclosed system may be configured to make certain therapeutic decisions related to user-defined set points and measured physiological parameters. For example, the disclosed system may be configured to recirculate CSF and infuse an exogenous fluid while monitoring ICP, and intermittently drain CSF from the recirculation loop as needed to maintain ICP according to a user-defined range or set point.

In certain embodiments, the disclosed system may comprise, in operable communication, any combination of the following subassemblies: pump mechanism(s), heating and/or cooling element(s), filtration element(s), flowrate control actuator(s), user interface(s), flow shutoff actuator(s), and the like.

In certain embodiments, the disclosed system may comprise, in operable communication, various sensing modalities, such as: pressure sensor(s), orientation sensor(s), acceleration sensor(s), motion sensor(s), flow sensor(s), bubble sensor(s), fluid level sensor(s), optical sensor(s), temperature sensor(s), electrochemical sensor(s), analyte sensors, and the like.

In various embodiments, sensors may be located within the patient interface assembly. In other embodiments, sensors may be located within the control system assembly, or provided as a separate assembly that may be durable or disposable depending on the type and construction of the specific sensor.

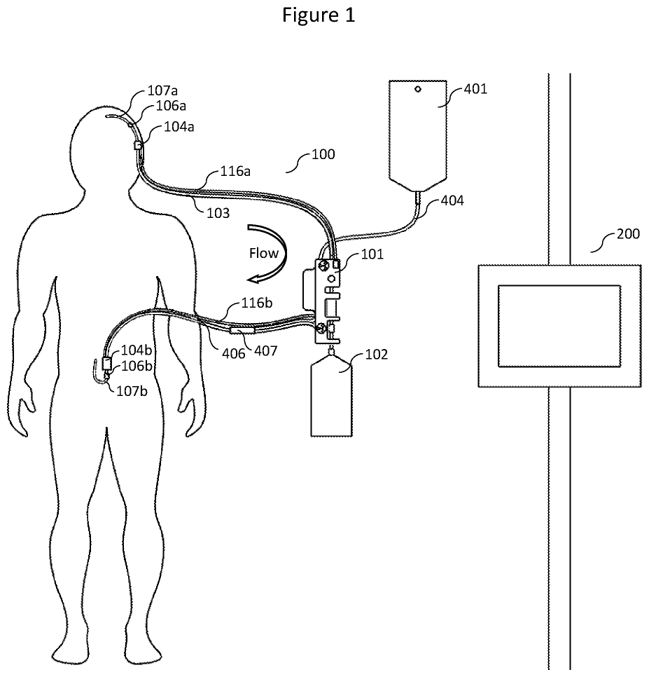

FIG. 1 is a line drawing showing a simplified diagram of a representative embodiment of the disclosed system as it relates to patient anatomy, wherein the system is configured for recirculation and drainage of CSF, infusion of a therapeutic fluid, and monitoring and control of various physiological parameters.

As depicted in FIG. 1, the disclosed system may comprise control system assembly 200 and patient interface assembly 100. Patient interface assembly 100 may comprise cartridge subassembly 101 for interfacing with the control system assembly; a body fluid recirculation loop comprising body fluid withdrawal tubing 103 and re-infusion tubing 406, which may also be called bi-directional tubing, for withdrawal of body fluid at one anatomical location and re-infusion of body fluid at the same anatomical location or a different anatomical location; body fluid collection reservoir 102 for permanent removal of body fluid from the recirculation loop; wearable sensor subassemblies 104a and 104b configured for attachment to the patient proximate to a suitable anatomical marker such as the external auditory meatus (EAM) or L4/L5 spinous process; electrical cables 116a and 116b for electrical communication and DC power distribution between said cartridge subassemblies and said wearable sensor subassemblies; fluid-tight connectors 106a and 106b for connection to implanted catheters 107a and 107b (such as ventricular catheters, parenchymal catheters, lumbar catheters, etc.).

In certain embodiments, the disclosed system may additionally comprise exogenous fluid reservoir 401 and exogenous fluid tubing 404 for infusion of exogenous fluid and mixing chamber 407 for mixing of exogenous fluid with fluid in the body fluid recirculation loop.

In other embodiments, said electrical cables 116a and 116b may be replaced with battery power and/or one or more wireless communication protocol(s) (e.g., Wi-Fi, BLE, IR, etc.).

In certain embodiments, the disclosed system may comprise more than one patient interface assembly 100.

As depicted in FIG. 1, recirculation flow of CSF may be directed externally from an anatomical location in the head (e.g., the ventricles of the brain, the subarachnoid space, etc.) to an anatomical location in the spine (e.g., L4/L5, C1/C2, etc.). In related embodiments, recirculation flow of CSF may be reversed by swapping the connections between the body fluid withdrawal and re-infusion tubing, and the implanted catheters, such that fluid-tight connector 106a is connected to implanted catheter 107b and fluid-tight connector 106b is connected to implanted catheter 107a. In other embodiments, fluid connections and implanted catheters may be at locations other than those shown herein. For example, fluid may be withdrawn from the ventricles of the brain and re-infused into the brain parenchyma or may be removed from the thoracic region of the spine and re-infused into the lumbar region of the spine. In certain embodiments, the disclosed system may be configured to remove CSF from an anatomical location, store it temporarily extracorporeally, and then return it to the same anatomical location for therapeutic or diagnostic benefit.

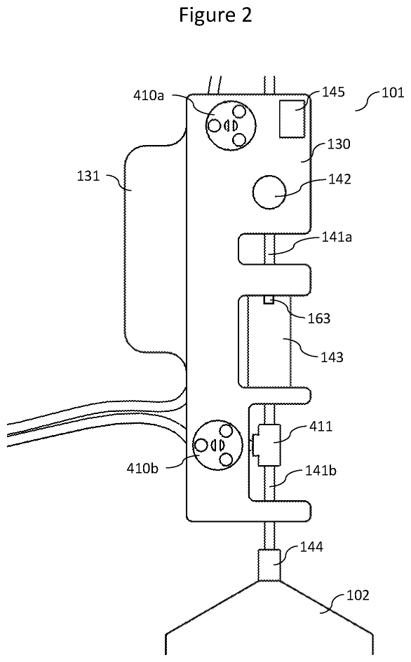

FIG. 2 is a line drawing showing a detailed view of a representative embodiment of cartridge subassembly 101.

As depicted in FIG. 2, cartridge subassembly 101 may comprise cartridge housing 130 with grip feature 131 for inserting and removing the cartridge subassembly into/from said control system assembly.

Cartridge subassembly 101 may further comprise first compliant tubing 141a, which may be variably pinched or released by a first flow control mechanism in the control system assembly to disallow or allow gravity-based drainage of a target body fluid into drip chamber 143, tee-fitting 411, and second compliant tubing 141b, which may be variably pinched or released by a second flow control mechanism in the control system assembly to allow gravity based drainage of said fluid from said drip chamber into drainage collection reservoir 102 when released for permanent removal from the recirculation loop.

Said drip chamber may comprise drip orifice 163, wherein said drip orifice 163 produces drips of a known size, and may interface with a drip detector in the control system assembly. Flowrate may be computed by the system by counting the number of drips detected by a corresponding drip detector in the control system assembly and multiplying the number of drips by the known drip size.

Cartridge subassembly 101 may further comprise flow shutoff feature with attached button 142 wherein flow shutoff feature may be momentarily disengaged by depressing said button to allow body fluid drainage.

Cartridge subassembly 101 may further comprise fluid-tight fitting 144 for optional connection, removal, and/or replacement of drainage reservoir 102.

Cartridge subassembly 101 may further comprise electrical contact array 145 to interface with a corresponding array in the control system assembly for electrical communication and DC power distribution between said control system assembly and said cartridge subassembly.

In certain embodiments, cartridge subassembly 101 may include first peristaltic pump head 410a to control flow from the exogenous fluid reservoir into the mixing chamber described elsewhere herein, and second peristaltic pump head 410b to control flow from the drip chamber back into the body. Said first and second peristaltic pump heads may interface with pump motors in the control system assembly. In other embodiments, certain aspects of said peristaltic pump assemblies depicted as being located in the cartridge subassembly may alternatively be located in the console assembly, or vice versa. In yet other embodiments, the described pumping action may be accomplished via an entirely different means (piezoelectric pump, diaphragm pump, etc.).

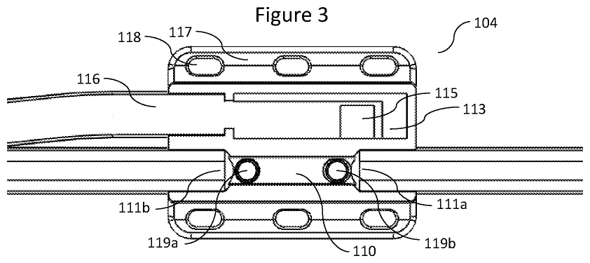

FIG. 3 is a line drawing showing a representative embodiment of wearable sensor subassembly 104 (previously depicted as 104a and 104b in FIG. 1).

As depicted in FIG. 3, wearable sensor subassembly 104 may comprise flow channel 110 with fluid inlet 111a and fluid outlet 111b opposite the fluid inlet, pressure sensors 119a and 119b for measuring fluid pressure, and orientation sensor(s) 115 (e.g., accelerometer, gyroscope, etc.) for detecting patient movement. Wearable sensor subassembly 104 may further comprise housing 117 with suture points 118 for attaching wearable sensor subassembly 104 to a patient near a suitable anatomical marker as described elsewhere herein. Number and position of said suture points may vary based on the anatomical marker. Sensors and associated components may be co-located on PCB 113 (comprising a substrate of FR4, Kapton, polyester, etc.) and may utilize a single communication and power interface along electrical cable 116. In other embodiments, sensors may be located at any other suitable position along the fluid line (e.g., within the cartridge, between the cartridge and the drainage reservoir, etc.). In yet other embodiments, certain sensors that do not directly contact the fluid in the drainage or recirculation fluid line(s) may be located within the control system assembly or provided as separate assemblies that may be used in operable communication with the control system assembly.

In other embodiments, said electrical cable may be replaced with battery power and/or one or more wireless communication protocol(s) (e.g., Wi-Fi, BLE, etc.).

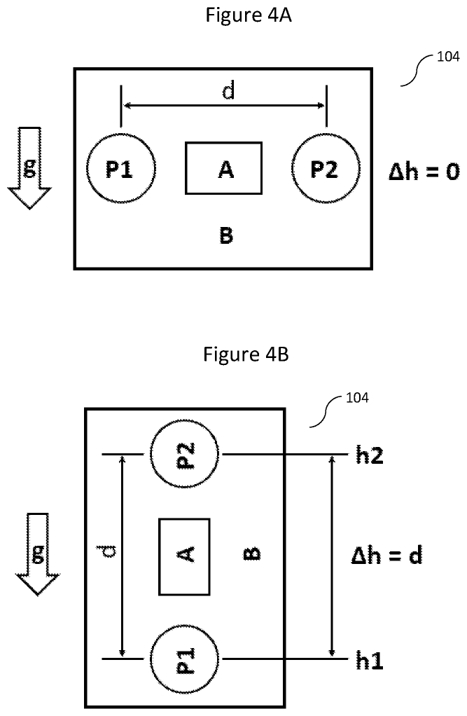

FIG. 4A and FIG. 4B are schematic representations of wearable sensor subassembly 104 according to certain embodiments of the present disclosure, wherein an orientation sensor (A) and a plurality of pressure sensors (P1 and P2) are mounted at a fixed spacing distance (d) onto a rigid or semi-rigid member (B). FIG. 4A depicts wearable sensor subassembly 104 in a horizontal orientation with respect to gravity vector (g). FIG. 4B depicts wearable sensor subassembly 104 in a vertical orientation with respect to gravity vector (g).

Within certain aspects of this embodiment, the orientation sensor(s) detects the orientation of wearable sensor subassembly 104 thereby facilitating calculation of an anticipated pressure differential ΔP according to the formula:

ΔP_{anticipated}=ρ(Δh)
wherein ρ is the fluid density (e.g., the density of CSF, saline, blood, urine, exogenous fluid, etc.) and Δh is the height differential between pressure sensors P2 and P1 with respect to the gravity vector.

As depicted in FIG. 4A, when wearable sensor subassembly 104 is oriented horizontally with respect to the gravity vector (g), the pressure readings of the plurality of pressure sensors (P1 and P2) are substantially equivalent because the height differential (Δh) between the two pressure sensors (P1 and P2) is zero (i.e., Δh=0, so ΔP_{anticipated}=0).

As depicted in FIG. 4B, when wearable sensor subassembly 104 is oriented vertically with respect to the gravity vector (g), the pressure differential between the plurality of pressure sensors (P1 and P2) is maximized because the height differential (Δh) between the two pressure sensors is also maximized (i.e., Δh=d, so ΔP_{anticipated}=ρd).

In any wearable sensor subassembly orientation other than horizontal or vertical, the height differential between the plurality of pressure sensors (P1 and P2) will vary between 0 and d based on the vertical component of orientation with respect to the gravity vector (g). The corresponding anticipated pressure differential will range from ΔP_{anticipated}=0 to ΔP_{anticipated}=ρd.

In certain embodiments of the control system algorithm, one or both pressure sensors may be used to determine actual measured fluid pressure, while any substantial deviation between ΔP_{anticipated} (as described above) and ΔP_{actual} (obtained directly via pressure sensor readings) may be used by the system to detect pressure sensor faults (electrical failure, drift in sensor accuracy, biofouling, etc.).

It will be apparent to one skilled in the art that the current disclosure is applicable to the measurement of gauge or absolute pressure, since either may be accomplished depending on the type of sensor used for P1 and P2, or the inclusion of separate atmospheric pressure sensor(s) outside the fluid path (such as in the control system assembly) for the calculation of gauge pressure.

The disclosed approach provides two layers of redundancy. Firstly, since each pressure sensor in wearable sensor subassembly 104 is located proximate to an anatomical marker for the fluid of interest, a second pressure sensor provides a direct “backup” that may allow the system to continue operating in the event that either sensor is determined to no longer be functioning normally. Secondly, the system may detect very small amounts of drift in the accuracy of the wearable sensor subassembly and take appropriate action (such as notifying the user) before such errors become clinically relevant.

The disclosed approach differs from existing two-sensor systems, wherein one sensor measures the pressure in the target fluid line and a second sensor measures the pressure in a separate reference line, and wherein both pressure sensors are positioned at a location other than a relevant anatomical marker (e.g., in a pole-mounted console or hip-worn wearable). In such systems, the true pressure of the target fluid (e.g., true ICP) is calculated as the difference between the pressure in a drain line and the pressure in a separate reference line.

Two-sensor arrangements described in the prior art provide no redundancy and limited opportunities for error-checking, leaving the patient vulnerable to sensor drift and similar faults. The co-location of two pressure sensors and an orientation sensor substantially proximate to a relevant anatomical marker as described in the current disclosure provides an unprecedented level of measurement accuracy and clinical safety.

It will be appreciated that wearable sensor subassembly 104 must be sufficiently small and lightweight to facilitate attachment to certain anatomical markers (such as the EAM, which is located on the head, or L4-L5 located in the lumbar region of the lower back) in order to achieve practical use. As such, the use of sufficiently small pressure sensors, which are suitable for extended contact with body fluids, and which are also of sufficient accuracy and precision as to enable clinical utility, is critical to achieving the disclosed embodiments. Furthermore, the spacing distance between the sensors must be sufficiently small as to facilitate a suitable overall footprint for the assembly, which places further constraints on the precision of said pressure sensors to enable useful drift detection as described elsewhere herein. For example, a spacing distance on the order of a few centimeters is only useful if the pressure sensors are able to resolve pressure differences on the order of a few millimeters of water (mmH2O). Such pressure sensors were unknown to the art until recently, rendering such embodiments impractical. However, due to recent technological developments, spacing distances (d) in the range of 1-2 cm are now possible, using tiny (2-3 mm wide) pressure sensors with precision on the order of ±1 mmH2O, enabling practical embodiments of wearable sensor subassemblies with an overall footprint in the range of 2-5 cm² that have the characteristics described herein.

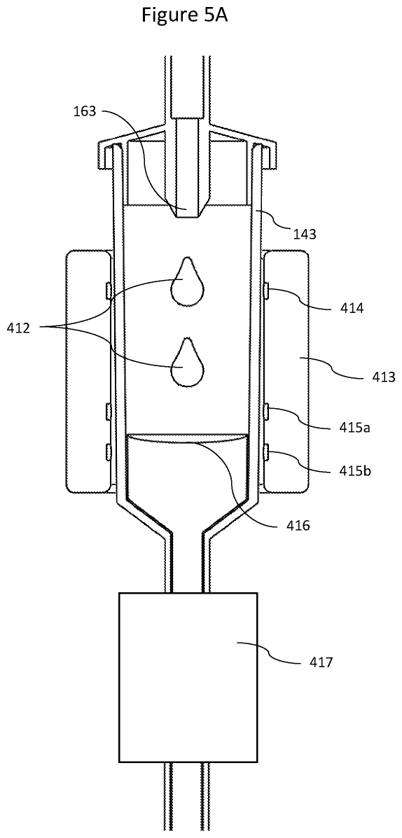

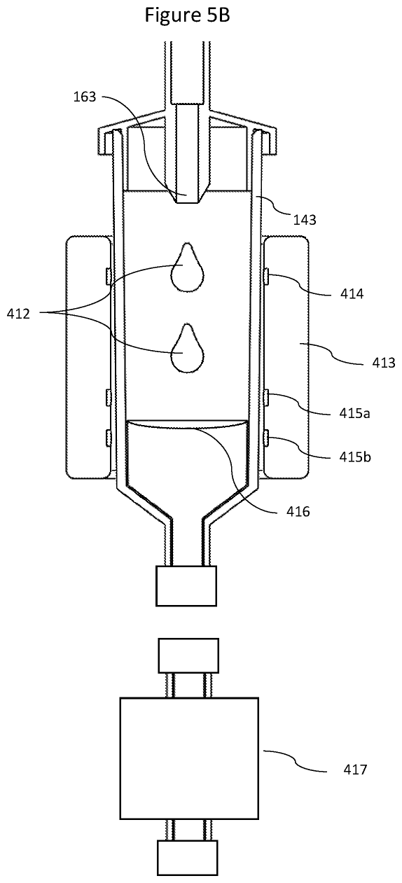

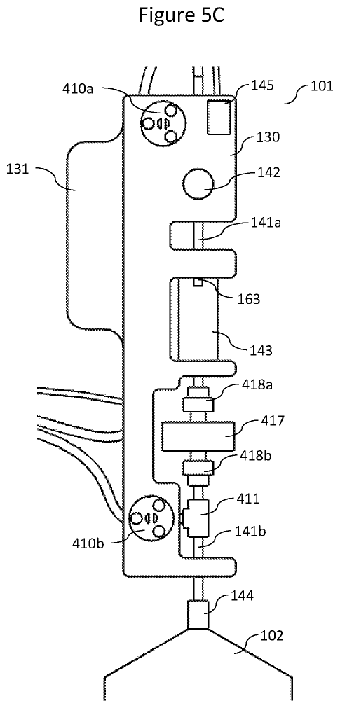

FIGS. 5A and 5B are line drawings showing a section view of representative embodiments of the drip and fluid level detection interface between the cartridge subassembly and the control system assembly.

As depicted in FIGS. 5A and 5B, the cartridge subassembly may comprise drip chamber 143 with drip orifice 163, which produces fluid drips 412 of a known size. The control system assembly may comprise fluid detection subassembly 413 that interfaces with said drip chamber. Fluid detection subassembly 413 may comprise one or more drip sensor(s) 414 for detecting fluid drips 412 and one or more fluid level sensors 415a and 415b for detecting fluid level 416 accumulated in said drip chamber. Fluid detection subassembly 413 may also be referred to as a drip and fluid level sensor, a body fluid flow measurement sensor, etc.

In other embodiments, drip detection and fluid level detection may be accomplished by two (or more) separate sensors rather than the combined sensor depicted in FIGS. 5A and 5B. In certain embodiments, drip and fluid level sensor(s) may be optical (infrared, etc.), ultrasonic, or any other suitable sensing technology.

In other embodiments, said drip and fluid level sensor assembly may additionally comprise optical sensor(s) to monitor color, opacity, or other parameter(s) that may indicate a corresponding change in viscosity or density of the fluid, which may affect the actual volume of fluid contained in each drip. In such cases, the disclosed system may comprise an algorithm to compensate for said changes in drip volume to maintain accurate measurement of flowrate.

In other embodiments, as depicted in FIG. 5A, the cartridge subassembly may further comprise filter 417 for removing biologic or non-biologic material from withdrawn fluid. Said filter may be mechanical (e.g., a porous membrane such as paper, ePTFE, sintered metal or other media or a mechanical separator such as centrifuge), electrochemical, electrostatic, or any other means suitable for selective filtration of the target material to be removed. Filter 417 may be removable and replaceable as depicted in FIG. 5B.

FIG. 5C is a line drawing showing a detailed view of a representative embodiment of cartridge subassembly 101 comprising filter 417. As shown in FIG. 5C, cartridge subassembly 101 may additionally comprise filter 417, which may be removable and replaceable via fluid-tight fittings 418a and 418b. Filter 417 may be located between the outflow of drip chamber 143 and the inflow of tee-fitting 411. In alternate embodiments, filter 417 may be located anywhere along re-infusion tubing 406.

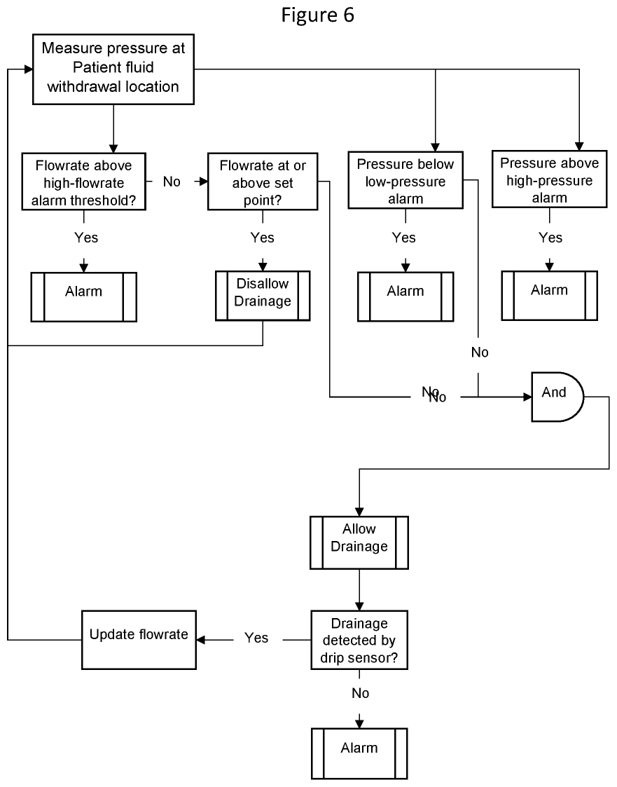

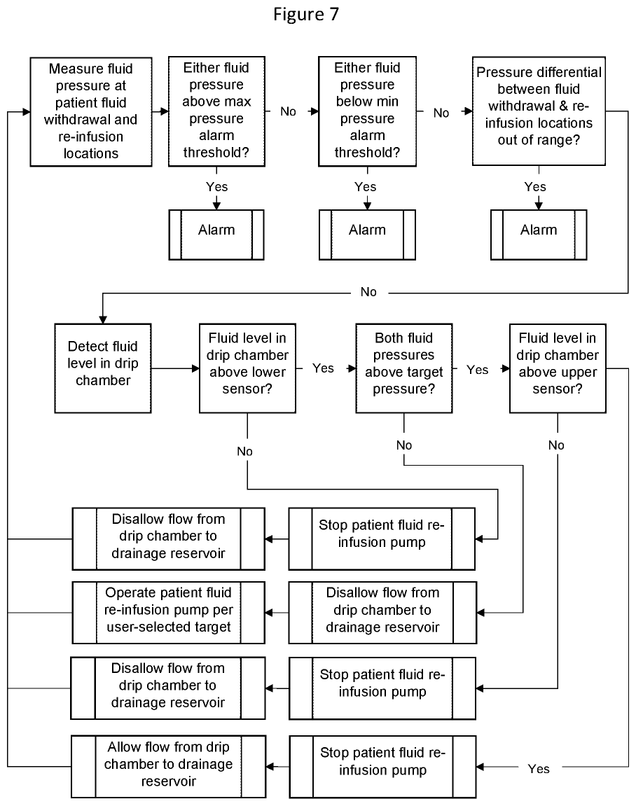

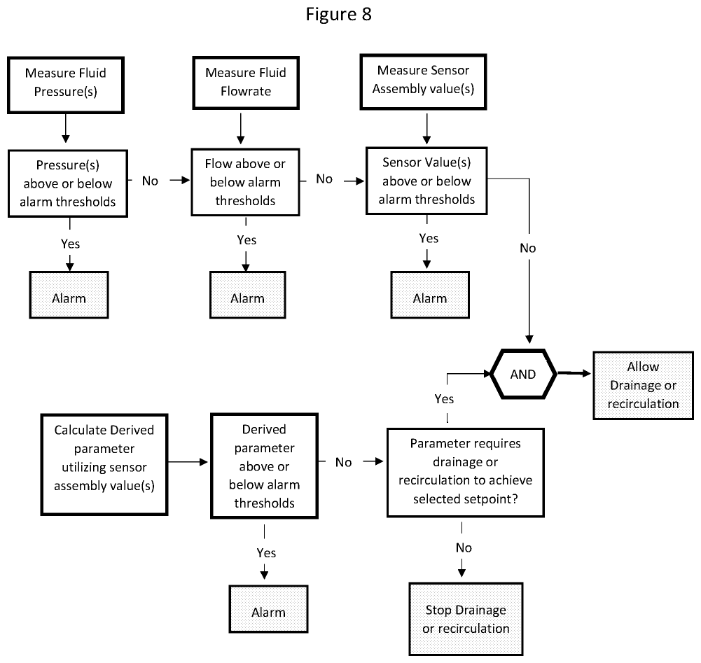

FIG. 6, FIG. 7 and FIG. 8 are block diagrams depicting simplified representative algorithms that may be employed by the disclosed system. The system may comprise a plurality of operating modes depending on the type of therapy being delivered and/or the sensing modalities employed, and the algorithm(s) may therefore differ from those shown in FIG. 6 or FIG. 7 or FIG. 8 in various ways without departing from the spirit of the disclosure.

As depicted in FIG. 6, the disclosed system may be configured to provide controlled therapeutic drainage of a body fluid according to a user-defined flowrate or pressure set point while monitoring user-defined alarm threshold values for parameters such as pressure and flowrate. In certain embodiments, the system may be further configured to monitor the output of one or more sensors for the detection of one or more analytes or contaminants associated with disease, toxicity, and/or infection, and to assert alarm(s) or notification(s) to the user when such analytes, contaminants, or toxins are detected. In certain embodiments the system may be further configured to utilize algorithms(s) derived from human analysis and analytics, machine learning, neural network(s), artificial intelligence, or similar methods, to deliver more nuanced and personalized management of certain medical conditions, specific physiological parameters, and the like.

In certain embodiments, as depicted in FIG. 8, the disclosed system may be configured to provide controlled therapeutic recirculation of a body fluid according to algorithm(s) or user-defined targets utilizing the detection or concentration of the analytes, contaminants, or toxins. For example, in the case of a subdural bleed or subarachnoid hemorrhage, the disclosed system may measure blood concentration in the CSF continuously or at defined intervals and drain CSF whenever the concentration exceeds an acceptably low level. The detection of the concentration of blood may be measured by an analyte sensor or by a physiological sensor optically evaluating the color of the CSF or identifying fluid composition using photo sensing at various wavelengths.

The disclosed system may additionally be configured to automatically manage fluid levels in the drip chamber, utilizing an algorithm such as that depicted in FIG. 7. The system may variably operate one or more mechanism(s), such as peristaltic pump(s), pinch valve(s), etc. to control flow of a drained body fluid and/or an exogenous fluid either back into the patient from which said drained body fluid was withdrawn as a form of autotransfusion, or into a drainage collection reservoir for permanent removal of said body fluid from the recirculation loop, according to various user-defined parameters such as target pressure and/or flowrate, allowable ranges of pressure and/or flowrate, and/or alarm thresholds for pressure and/or flowrate.

Control system assembly 200 may include one or more processors (microcontroller, microprocessor, FPGA, SOM, etc.) and one or more storage devices (EEPROM, flash memory, etc.) storing instructions that, when executed by the one or more processors, cause control system assembly 200 to perform the algorithms described herein.

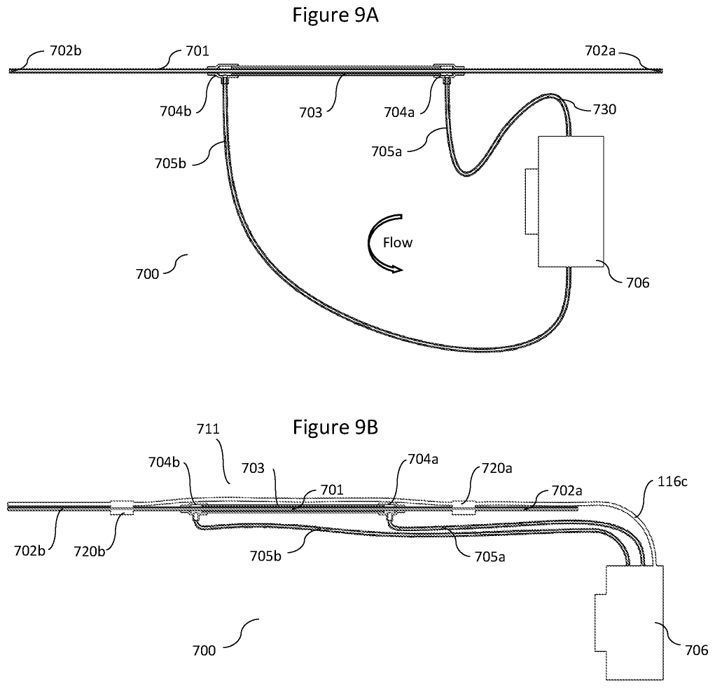

FIG. 9A is a line drawing showing a representative embodiment of a thermal conditioning assembly which may be included in the disclosed system. As depicted in FIG. 9A, thermal conditioning assembly 700 may comprise patient fluid line 701 with patient fluid inlet 702a and patient fluid outlet 702b opposite the fluid inlet, thermal conditioning cartridge subassembly 706 and thermal transfer subassembly 711 comprising thermal conditioning jacket 703 with working fluid inlet port 704a and working fluid outlet port 704b, working fluid inflow tube 705a and working fluid outflow tube 705b containing working fluid 730.

In certain embodiments, said thermal conditioning cartridge subassembly may comprise a heating or cooling element (e.g., a cartridge heater, thermoelectric cooler, etc.) to heat or cool the working fluid and a pump or similar means to circulate the working fluid around the working fluid loop. Said working fluid may provide thermal exchange between the patient fluid and the working fluid to accomplish heating or cooling of the patient fluid. In related embodiments, said cartridge subassembly may interface with a durable assembly (such as a console assembly) that may provide power, thermal conditioning, pumping, etc. either directly or indirectly to the working fluid.

Thermal conditioning assembly may further comprise temperature measurement subassembly 720, said temperature measurement subassembly comprising a temperature sensor, thermocouple, or other suitable temperature measurement device for monitoring the temperature of the fluid in the patient fluid line. In other embodiments, the wearable sensor subassembly described elsewhere herein may comprise said temperature measurement subassembly.

Working fluid inlet and outlet ports may be made of a rigid (plastic, metal, ceramic, etc.) or flexible (silicone, TPE, etc.) material depending on the configuration of the disclosed system and the anatomical location in which it is used clinically. For certain applications (e.g., cranial therapies, neonatal, etc.) the patient fluid tubing, working fluid tubing, fittings, etc., may be sufficiently small and flexible to be constrained to the patient anatomy without imparting undue forces (e.g., the weight of the tubing) to the patient anatomy.

Patient fluid tubing and working fluid tubing may be made of a compliant or semi-compliant material (silicone, TPE, polyurethane, pebax, etc.) depending on the configuration of the disclosed system and the anatomical location in which it is used clinically. Said tubing may be constrained to the patient anatomy by sutures, a wearable device, or any other suitable means.

Working fluid may be any fluid suitable for thermal transfer (e.g., water, liquid silicone, mineral oil, etc.).

FIG. 9B is a line drawing showing another representative embodiment of a thermal conditioning assembly 700. As depicted in FIG. 9B, thermal conditioning assembly 700 may additionally comprise thermal monitoring inflow sensor 720a and/or thermal monitoring outflow sensor 720b to provide electrical signals corresponding to the temperature of the patient fluid, which may be interpreted by a thermal control subassembly in the control system assembly. Said thermal control subassembly may comprise one or more logic device(s) (such as a microcontroller, microprocessor, or the like) which may implement one or more control algorithm(s) to adjust the temperature and/or flowrate of the working fluid, thereby adjusting the thermal transfer rate between the working fluid and the patient fluid, based on the electrical signals from thermal monitoring inflow sensor 720a and/or thermal monitoring outflow sensor 720b. Electrical signals from thermal monitoring inflow sensor 720a and/or thermal monitoring outflow sensor 720b may be transmitted via electrical cable 116c to thermal conditioning cartridge subassembly 706 or control system assembly 200, or said electrical signals may be transmitted wirelessly (e.g., via Wi-Fi, BLE, IR etc.) and may be used by control system assembly 200 as a input for patient fluid flowrate control.

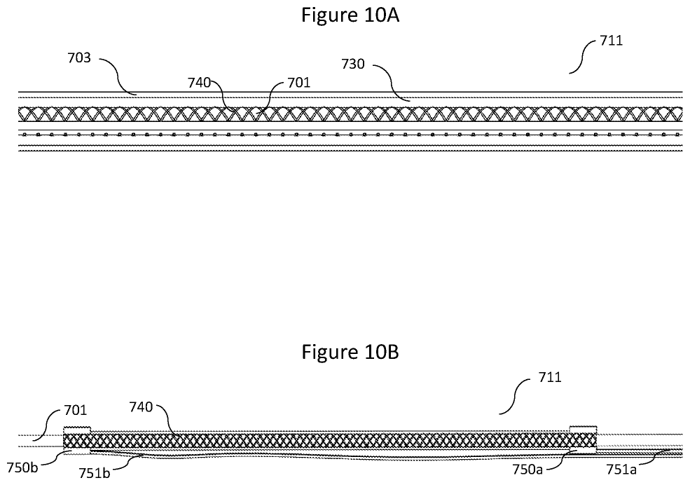

FIGS. 10A and 10B are line drawings showing alternate embodiments of thermal transfer subassembly 711. FIG. 10A depicts a partial cutaway view of a section of patient fluid line 701 enclosed by thermal conditioning jacket 703. As depicted in FIG. 10A, a portion of patient fluid line 701 may comprise wire braid 740 embedded into or onto its wall to enhance heat transfer between working fluid 730 and patient fluid. In alternate embodiments, wire braid 740 may be configured as one or more helically-wound wire(s), straight wires, wire rings, or wire mesh. In yet other embodiments, wire braid 740 may be replaced by, or used in conjunction with, thermally-conductive fillers or fibers (e.g., carbon, metallic spheres or flakes, etc.) comprised directly into the material of patient fluid line 701.

As depicted in FIG. 10B, a portion of patient fluid line 701 may comprise wire braid 740 embedded into or onto its wall, wherein wire braid 740 may be electrically connected to electrical interface 750a and 750b, which may in turn be connected to electrical wires 751a and 751b, wherein wire braid 740 may be heated directly by passing current between wires 751a and 751b. In alternate embodiments, wire braid 740 may be configured as one or more helically-wound wire(s), straight wires, or wire mesh.

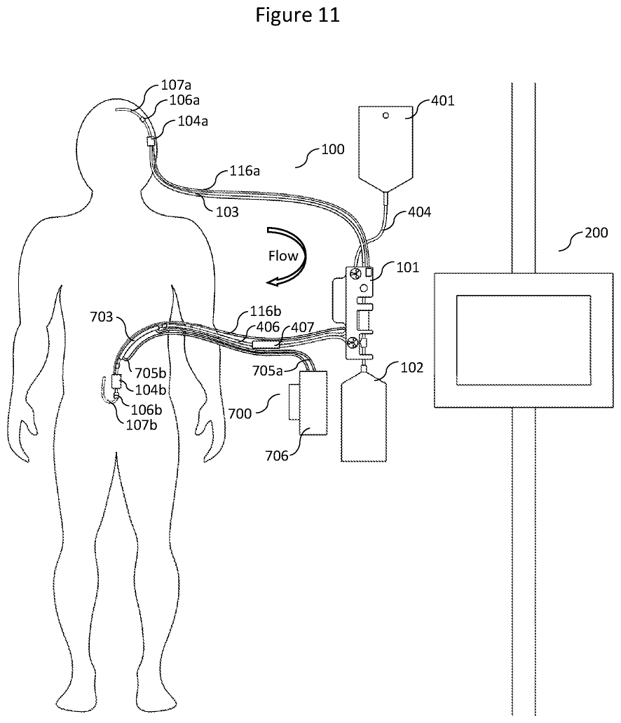

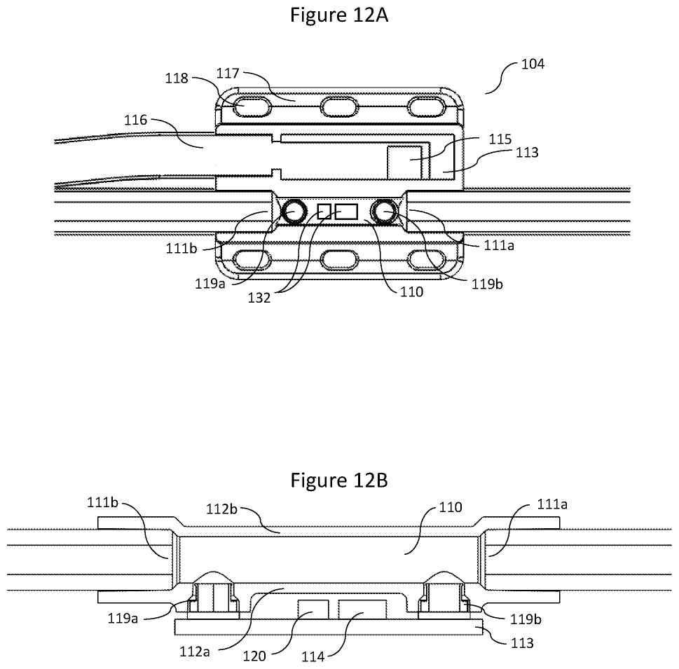

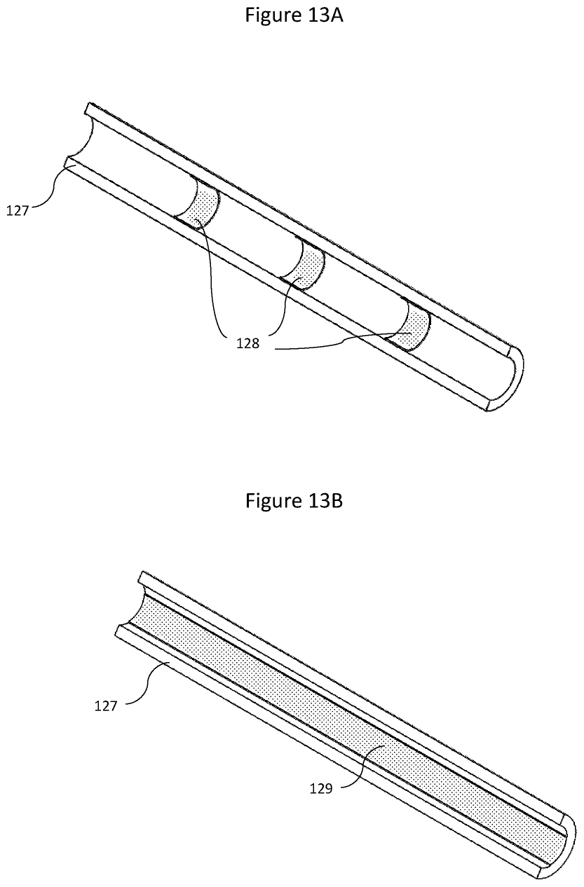

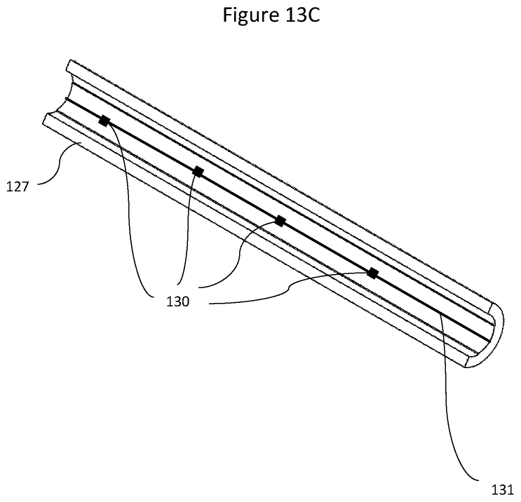

As depicted in FIG. 11, the disclosed system may comprise control system assembly 200 and patient interface assembly 100. Patient interface assembly 100 may further comprise thermal conditioning assembly 700 as described elsewhere herein. The disclosed system may further comprise one or more sensors as depicted in FIGS. 12A and 12B, and FIGS. 13A, 13B and 13C. Some sensors may be fluid communicating while others may not. As depicted in FIGS. 12A and 12B, these sensors may be co-located on PCB 113 (comprising a substrate of FR4, Kapton, polyester, etc.) of wearable sensor subassembly 104, and may comprise one or more optical sensor 114, orientation sensor 115, pressure sensors 119a and 119b, and/or fluid communicating sensors 132.

In related embodiments, wireless technologies may be used to accomplish transmission of power (inductive or resonant-inductive coupling, etc.) and/or signal communication (RF, infrared, etc.). In other related embodiments, wearable sensor subassembly 104 may be powered internally (battery, capacitor, etc.). In further embodiments, more than one wearable sensor subassembly may be located at any suitable location along body fluid withdrawal or drainage line 103 or re-infusion tubing 406

As depicted in FIGS. 13A, 13B, and 13C, fluid-communicating sensor(s) optimized for the detection of one or more of the analytes or contaminants described elsewhere herein, may be located directly on and/or in fluid line 127 such as body fluid withdrawal or drainage line 103 or re-infusion tubing 406 at any suitable location. FIG. 13A depicts the use of sensing bands 128 such as colorimetric coating sensors delivering visually detectable results directly to the user. FIG. 13B depicts the use of a continuous sensing strip 129, which may comprise colorimetric coating as described herein or it may comprise a flex circuit adhered to the inner surface of fluid line 127 or suspended within the body fluid of fluid line 127.

In other embodiments, fluid line 127 fabrication itself may comprise a sensing material such as a leuco dye, which can serve as a colorimetric sensor due to its chemical form changing property caused by changes in heat, light or pH. In related embodiments, as depicted in FIG. 13C, electronic fluid-communicating sensors 130 may be embedded in the fluid line itself using overmolding, co-extrusion or other fabrication methods and an electrical cable 131 or flat flex circuit that may be fabricated within the tubing material and/or attached to the inside or outside of fluid line 127.

In other embodiments, sensors and/or illumination sources may be located at any other suitable position along the fluid line (e.g., within a second sensor assembly, within the cartridge, between the cartridge and the drainage reservoir, etc.). In yet other embodiments, sensors and/or their illumination source(s) that do not directly contact the fluid in the drainage line may be located within the control system assembly or provided as separate assemblies that may be used in operable communication with the control system assembly 200. In related embodiments, sensor(s) and/or illumination source(s) may be partially or fully enclosed in opaque or semi-opaque enclosure(s) to minimize and/or control interference from ambient light.

U.S. provisional patent application Nos. 63/315,910, filed Mar. 2, 2022, and 63/315,930, filed Mar. 2, 2022, to which this application claims priority, are hereby incorporated herein by reference in their entireties. Aspects of the various embodiments described above can be combined to provide further embodiments. In general, in the following claims, the terms used should not be construed to limit the claims to the specific embodiments disclosed in the specification and the claims, but should be construed to include all possible embodiments along with the full scope of equivalents to which such claims are entitled.

1. A body fluid management system configured for recirculation and drainage of a body fluid, said body fluid management system comprising:
a patient interface assembly including a first fluid line, a second fluid line, a body fluid collection reservoir, and at least one wearable sensor subassembly, wherein said first fluid line is configured for connection to a first implanted catheter at a first patient access location and said second fluid line is configured for connection to a second implanted catheter at a second patient access location; and
a control system assembly including at least one fluid detection subassembly, at least one logic device, and a plurality of flow control mechanisms;
wherein said logic device implements an algorithm to control operation of said plurality of flow control mechanisms based on inputs from at least one of said fluid detection subassembly and said wearable sensor subassembly;
wherein said plurality of flow control mechanisms are configured to variably withdraw said body fluid from said first patient access location, re-infuse said body fluid at said second patient access location, and drain said body fluid into said body fluid collection reservoir.

2. The system of claim 1 wherein said patient interface assembly includes a third fluid line configured at one end for connection to an exogenous fluid source containing an exogenous fluid. 3 The system of claim 2 wherein said patient interface assembly includes a mixing chamber configured for mixing said exogenous fluid and said body fluid prior to re-infusion at said second patient access location.

4. The system of claim 1, wherein said wearable sensor subassembly comprises at least one pressure sensor in direct fluid communication with said body fluid and wherein said control system assembly is configured for monitoring changes in a pressure of said body fluid.

5. The system of claim 1 wherein said wearable sensor subassembly comprises at least one analyte sensor in direct fluid communication with the body fluid and wherein said control system assembly is configured for monitoring changes in a concentration of a target analyte in said body fluid.

6. The system of claim 1 wherein said wearable sensor subassembly comprises at least one physiological sensor and wherein said control system assembly is configured for monitoring changes in a physiological parameter.

7. The system of claim 1 wherein a method of withdrawal of said body fluid is gravity-based drainage.

8. The system of claim 1 wherein said body fluid is cerebrospinal fluid.

9. The system of claim 1 wherein said first and second patient access locations are proximate to a single anatomical marker, and wherein said at least one wearable sensor subassembly is configured for attaching to the patient proximate to said single anatomical marker.

10. The system of claim 9 wherein said anatomical marker is an external auditory meatus.

11. The system of claim 1 wherein said first patient access location is proximate to a first anatomical marker and said second patient access location is proximate to a second anatomical marker.

12. The system of claim 11 wherein said at least one wearable sensor subassembly comprises a first wearable sensor subassembly and a second wearable sensor subassembly, wherein said first wearable sensor subassembly is configured for attaching to the patient proximate to said first anatomical marker and said second wearable sensor subassembly is configured for attaching to the patient proximate to said second anatomical marker.

13. The system of claim 11 wherein said first anatomical marker is an external auditory meatus and said second anatomical marker is a lumbar region of the spine.

14. A body fluid management system configured for conditioning and recirculation of a body fluid, said body fluid management system comprising:
a patient interface assembly including a first fluid line, a second fluid line, at least one wearable sensor subassembly, and at least one body fluid conditioning assembly, wherein said first fluid line is configured for connection to a first implanted catheter at a first patient access location and said second fluid line is configured for connection to a second implanted catheter at a second patient access location; and
a control system assembly including at least one fluid detection subassembly, at least one logic device, and a plurality of flow control mechanisms;
wherein said logic device implements an algorithm to control operation of said plurality of flow control mechanisms based on inputs from at least one of said fluid detection subassembly and said wearable sensor subassembly;
wherein said plurality of flow control mechanisms are configured to variably withdraw said body fluid from said first patient access location, re-infuse said body fluid at said second patient access location, and drain said body fluid into said body fluid collection reservoir.

15. The system of claim 14 wherein said body fluid conditioning assembly is a thermal conditioning assembly and said thermal conditioning assembly is configured to adjust a temperature of said body fluid in said second fluid line upstream of said second patient access location.

16. The system of claim 15 wherein said thermal conditioning assembly is configured to increase the temperature of said body fluid.

17. The system of claim 15 wherein said thermal conditioning assembly is configured to decrease the temperature of said body fluid.

18. The system of claim 15 wherein said patient interface assembly includes at least one temperature sensor configured to measure the temperature of said body fluid, and said control system assembly is configured to control operation of said thermal conditioning assembly based on inputs from said temperature sensor.

19. The system of claim 15 wherein said control system assembly is configured to receive body temperature input and adjust an output of said thermal conditioning assembly based on said input.

20. The system of claim 14 wherein said body fluid conditioning assembly is a filtration assembly configured for capturing one or more contaminants from said body fluid.

21. The system of claim 14 wherein said body fluid is cerebrospinal fluid.

22. A body fluid management system configured for recirculation and drainage of a body fluid, said body fluid management system comprising:
a patient interface assembly including a bi-directional body fluid line, a body fluid collection reservoir, and at least one wearable sensor subassembly, wherein said fluid line is configured for connection to an implanted catheter at a patient access location; and
a control system assembly including at least one fluid detection subassembly, at least one logic device, and a plurality of flow control mechanisms;
wherein said logic device implements an algorithm to control operation of said plurality of flow control mechanisms based on inputs from at least one of said fluid detection subassembly and said wearable sensor subassembly;
wherein said plurality of flow control mechanisms are configured to variably withdraw said body fluid from said patient access location, temporarily store said body fluid, re-infuse said body fluid at said patient access location, and drain said body fluid into said body fluid collection reservoir.
